(19) United States
(12) Patent Application Publication
(10) Pub. No.: US 2025/0242343 A1
(43) Pub. Date: Jul. 31, 2025

(54) Solid Phase Reversible Immobilization (SPRI) Tips

(71) Applicant: DPX TECHNOLOGIES, INC., Columbia, SC (US)

(72) Inventors: William E. BREWER, Columbia, SC (US); Matthew G. FITTS, Columbia, SC (US); Paul MEEH, Columbia, SC (US)

(73) Assignee: DPX TECHNOLOGIES, INC., Columbia, SC (US)

(21) Appl. No.: 19/022,031

(22) Filed: Jan. 15, 2025

Related U.S. Application Data
(60) Provisional application No. 63/625,337, filed on Jan. 26, 2024.

(51) Int. Cl. B01L 3/02; C12N 15/10

(52) U.S. Cl. CPC B01L 3/0275; C12N 15/1003; B01L 2200/02; B01L 2200/0647; B01L 2300/042; B01L 2300/12

(57) ABSTRACT

A pipette tip for extracting nucleic acid, said pipette tip containing a glass plug made of a porous silica material with porosities of 5 to 20 μm and having a diameter of at least 0.8 mm and a thickness of at least 0.2 mm.

### PRIOR RELATED APPLICATIONS

This application claims priority to U.S. Ser. No. 63/625,337, filed Jan. 26, 2024, and incorporated by reference in its entirety for all purposes.

### FEDERALLY SPONSORED RESEARCH STATEMENT

Not applicable.

### REFERENCE TO MICROFICHE APPENDIX

Not applicable.

### FIELD OF THE DISCLOSURE

The disclosure relates generally to the field of chemical and biological sample preparation. Specifically, devices and methods for nucleic acid purification.

### BACKGROUND OF THE DISCLOSURE

Magnetic beads have become commonplace for nucleic acid purification, especially for next generation sequencing (NGS). The beads are made of paramagnetic materials, typically around 1 μm spherical particle size, that are coated with carboxylate groups or silica. A magnet is used to facilitate the extraction process, providing a means of separating the beads from various solutions, holding the beads in place, whilst solutions are exchanged.

The silanol or carboxylate groups are negatively charged in the presence of neutral aqueous solutions. By combining polyethylene glycol (PEG) with salts, such as sodium or magnesium, a salt bridge can be formed facilitating the binding (or immobilization) of deoxyribonucleic acid (DNA) or ribonucleic acid (RNA) on the bead. After the nucleic acid is bound, it can be eluted with water or a buffer. By varying the ratios of PEG and salt concentrations, larger DNA fragments can be preferentially bound. This is referred to as size selection.

The extractions are conducted by first incubating the sample solution with the magnetic beads and binding buffer (which contains the PEG and salts) for a set period of time (e.g., 5 minutes). After equilibration, the sample solution is moved to a magnet (or alternatively, a magnet is moved to the sample solution) to hold the magnetic beads with bound analyte to the bottom or sides of the container. While the magnet is engaged, the supernatant is removed by aspirating with a pipette tip.

Next the magnet is disengaged, and wash solution (such as 80% ethanol in water) added. The magnetic beads and wash solution are mixed by aspirating and dispensing the solution with another pipette tip. The magnet is again engaged, and the wash solution removed. These wash steps may be repeated as necessary.

Finally, the analytes of interest are eluted by adding an elution solvent. The beads are mixed with the elution solvent, and the analyte released from the beads into the eluate. The eluate solution is aspirated while the magnet is engaged and dispensed into another container for subsequent analysis or further processing.

There are a few disadvantages to this method. First, the multiple movements of the magnet (or sample container) in and out of position adds time to the method, and can be a source of error. Multiple pipette tips are used for processing, and these tips can be costly for certain automated robotic liquid handlers. The mixing of the beads with the various solutions are not always sufficient for good efficacy, especially in wash procedures. Finally, the biggest disadvantage is the possibility that some beads are not removed from the final solution when the magnet is engaged, which can cause downstream analytical issues.

Thus, there exists a need for a more efficient method to extract nucleic acid from reaction mixtures, especially for nucleic acid procedures, such as NGS, PCR and the like. The ideal method would avoid the use of magnetic beads, be fast, and allow the use of robotic liquid handlers for high throughput processing.

### SUMMARY OF THE DISCLOSURE

Generally speaking, the invention relates to devices and methods that allow the treatment of samples without the need for magnetic beads to hold samples at desired times. Instead of using magnetic beads, sintered silica beads or glass microfibers (GMF) with porosities of 5-20 μm are placed into pipette tips, forming a “glass plug” of sorts. Control of binding or release is by changing the ionic strength of the various wash and elution solvents. DNA, for example, is known to bind to silica in a solution with high ionic strength. The highest DNA adsorption efficiencies occur in the presence of buffer solution with a pH at or below the pKa of the surface silanol groups.

The mechanism behind DNA adsorption onto silica is not fully understood; one possible explanation involves reduction of the silica surface's negative charge due to the high ionic strength of the buffer. This decrease in surface charge leads to a decrease in the electrostatic repulsion between the negatively charged DNA and the negatively charged silica. Meanwhile, the buffer also reduces the activity of water by formatting hydrated ions. This leads to the silica surface and DNA becoming dehydrated. These conditions lead to an energetically favorable situation for DNA to adsorb to the silica surface. Once all processing steps are complete, the DNA can be eluted using a low salt elution buffer or even distilled water.

However, merely including sintered silica beads or glass microfibers (GMF) in a pipette tip is insufficient for this type of functionality. The porosity must be calibrated to both allow reversible binding of DNA and high throughput at typical robotic liquid handler flow rates. The narrow ends of typical high throughput robotic pipette tips approach 0.2 mm in diameter, so having any material inside the tip would greatly restrict flow.

Through our proof of concept testing herein, we have determined that glass plugs having porosities of 5 to 20 μm, with a diameter greater than 0.5 mm, and a thickness between 0.2 to 2.5 mm allows both adequate binding and sufficient flow of nucleic acid samples that are relatively clean. Such samples include DNA from sequencing reactions, DNA or RNA from cell extracts, amplicons from PCR reactions, and the like. Whole blood, by contrast, clogs the tips of this design and would need large diameter glass plugs or pretreatment to reduce the debris levels. Thus, the invention provides pipette tips with glass plugs therein, as well as methods of sample treatment using such tips.

The silica material evaluated herein included sintered silica beads of approximately 100 μm, and glass microfibers (GMF) with porosities of 5 μm or greater. We have achieved slightly better recoveries of nucleic acid using GMF material, presumably due to higher surface area for active silanol groups. However, either material can be used.

As used herein, glass and silica are used interchangeably herein, each referring to a material with surface silanol groups that bind nucleic acid. The two major binding mechanisms are attractive interactions between phosphate and surface silanol groups and hydrophobic bonding between DNA/RNA base and silica hydrophobic region.

Although our focus herein is nucleic acid, particularly DNA, such devices may be used for any analyte that binds silica surfaces, or modified silica surfaces to create chemistries such as immunoaffinity and protein binding.

The invention thus includes one or more of the following embodiments in any combination(s) thereof:
A pipette tip for extracting nucleic acid, said pipette tip comprising: a hub that fits over a pipette head of a separate robotic liquid handler; said hub fluidly connected to a tapered portion fluidly connected to a delivery end; said tapered portion having friction fitted therein a glass plug having a diameter greater than 0.5 mm, and a thickness of between 0.2 to 2.5 mm; and said glass plug comprising a silica material having porosities of 5 to 20 μm.
A pipette tip for extracting nucleic acid, said pipette tip comprising: a hub that fits over a pipette head of a separate robotic liquid handler; said hub fluidly connected to a tapered portion fluidly connected to a delivery end; said tapered portion having friction fitted therein a glass plug having a diameter of about 0.8-1.2 mm, and a thickness of about 0.2-2.5 mm; and said glass plug comprising sintered glass beads or glass microfiber having porosities of 5 to 20 μm.
A method of extracting nucleic acid, the method comprising the steps of: a) preparing a sample by mixing salt and PEG with a solution comprising nucleic acid in a sample container and allowing said sample to equilibrate; b) aspirating said sample into any pipette tip herein described and dispensing said sample back into said sample container a plurality of times until said nucleic acid has bound to said silica material in said pipette tip, and finish by a last sample dispensing into said sample container; c) aspirating a wash solvent into said pipette tip and dispensing said wash solvent into a waste container; d) optionally repeating step c) a plurality of times; e) aspirating an elution solvent into said pipette tip and dispensing said elution solvent into a nucleic acid container; and optionally repeating step e a plurality of times.
Any pipette tip herein described, wherein said glass plug has a diameter of 0.8-1.2 mm.
Any pipette tip herein described, wherein said glass plug has a thickness of 0.2-2 mm.
Any pipette tip herein described, where said glass material is selected from common silicate glass, flint glass, soda lime glass, borosilicate glass, quartz glass, binder-free borosilicate glass, binder-containing borosilicate glass, or sintered glass beads or microfiber forms thereof.
Any pipette tip herein described, where said silica material comprises sintered glass beads.
Any pipette tip herein described, where said glass beads have diameters in the range of 20 to 500 μm, or a diameter of 50-400 μm.
Any pipette tip herein described, where said silica material comprises glass microfibers.
Any pipette tip herein described, where said silica material comprises glass microfibers of <20 μm diameter, preferably <10 μm or about 7.5 μm.
Any pipette tip herein described, wherein said silica material is supported on an annular ledge on an inside surface of said pipette tip.
Any pipette tip herein described, wherein said silica material is supported on an annular ledge with one or more cross-bracings on an inside surface of said pipette tip.
Any pipette tip herein described, wherein said silica material is contained inside a cylindrical cartridge housing said silica material.
Any pipette tip herein described, wherein said silica material is contained inside a tapered cylindrical cartridge housing said silica material.
Any method herein described, wherein said nucleic acid is DNA, or said sample is RNA.
Any method herein described, wherein said nucleic acid is a PCR amplicon.

As used herein, a “glass plug” or “glass section” refers to a body of material comprising sintered silica beads or silica microfibers having a porosity of about 5-20 μm, the plug having a diameter of at least about 0.5 mm and a thickness of about 0.2-2.5 mm. The term “plug” refers to the fact that there is solid material in the pipette tip, and is not intended to imply a lack of fluid flow. In fact, this plug allows sufficient flow for the tips to be used with robotic liquid handlers.

As used herein, a “microfiber” has fiber diameters less than twenty microns, and preferably less than 10 μm. The prototypes herein used GFM of 7.5 μm diameter.

As used herein, “glass microfiber” or “GMF” includes any form of glass fiber that is <20 microns in diameter that binds nucleic acids. Preferred glass fibers may include common silicate glass, flint glass, soda lime glass, borosilicate microfiber, quartz microfiber, binder-free borosilicate glass microfiber, and glass microfibers with a binder. The challenge in using GMF is to get reproducible amounts cut and reliably placed into the pipette tips. It is ideal for the GMF to be uniformly interconnected to allow for reproducible punching of the material. The preferred average diameter fiber sizes are from 1.0-10.0 μm.

As used herein “sintered glass beads” refers to glass beads, of the same glass types herein described, that have been sintered (heated) to cause the beads to soften and fuse, thus forming a very porous solid. When we discuss the porosity of the sintered glass herein, we refer to the porous disk formed by sintering and not the porosity of the individual beads.

Preferred sintered glass beads have diameters of 20-500 μm. The larger the sizes of the beads, the more porous. The more porous, the lower the recoveries with the highest flow rates. The smaller the diameters of the beads, the higher recoveries are achieved, but at the cost of poor flow rates. Thus, a balance is to be had, as herein taught.

The term “pipette tip” is a term of art, and refers to a conical tube with a larger proximal end, called the “hub” herein, and a narrow distal “delivery end” that is precisely engineered for accurate sampling and delivery of fluids, the body of the tip tapering between the hub and delivery end (“tapered portion”).

The hub fits over the “barrel” of a pipetting aid. Most manufacturers of handheld pipettes and robotic liquid handler systems make pipettes that will utilize universal tips, but this is not a requirement and there are other types of fit. The universal tip has a 1-2° taper for friction fit between the hub and the barrel and thus the interior diameter of the hub must be slightly larger than the barrel of the pipette and the inside taper (if present) of the pipette tip must also match the taper of the barrel. The fit is airtight, such that when e.g., the plunger of the pipette is pressed and released, a vacuum is applied, and fluid is pulled into the pipette tip. That fluid can be delivered or dispensed to any receptacle as needed by again depressing the plunger.

“Pipetting aids” include pipettors, micro-pipettors and robotic liquid handlers.

A “robotic liquid handler” is a robotic system, used for automation in chemical or biochemical laboratories that dispenses a selected quantity of reagent, samples or other liquid to a designated container. The simplest version can dispense an allotted volume of liquid from a motorized pipettor or syringe. More complicated systems can also manipulate the position of the dispensers and containers (often a Cartesian coordinate robot) and/or integrate additional laboratory devices or add-ons, such as microplate readers, heat sealers, heater/shakers, bar code readers, spectrophotometric or separation devices and instruments, storage devices, waste containers and incubators. In addition to the motorized pipettor or syringe, robotic liquid handlers also have trays for sample wells or sample vials, trays of pipette tips that fit the pipettor, and containers of solvents.

The methods described herein require a robotic liquid handler capable of manipulating the position of pipette tips on the Cartesian, 3-axis movements, typically implemented by means of an arm, and having multi-pipetting capabilities.

Exemplary robotic liquid handlers include the STAR, STARLET, or NIMBUS, each from HAMILTON®; Bravo Automated Liquid Handling Platform from AGILENT®; the epMotion from EPPENDORF®; the BIOMEK 4000 or NX or FX from BECKMAN COULTER®; the PIPETMAN from Gilson®; the FREEDOM EVO from TECAN®; PAL systems from CTC®; or the MPS from GERSTEL®, all of which are capable of being modified to perform pipetting and integrated with a variety of separation-mass spectrometric instruments. However, any commercially available robotic liquid handler can be used and/or modified to perform the disclosed separations.

The term “robotic pipette tip” is a pipette tip whose inner taper in the hub is such as to fit a robotic liquid handler. Most frequently there is no difference between a robotic pipette tip and a pipette tip for a hand-held micropipette, but there can be size differences. Most robotic liquid handlers use universal pipette tips with a 1-2° taper on the inner surface of the hub that matches with a 1-2 taper on the outer surface of the pipette head. However, some robotic liquid handlers use a tip with no taper (e.g., HAMILTON®) and other shapes are possible.

The term “wide bore pipette tip” refers to a pipette tip that is cylindrical through most of its length, rather than conical, with the orifice at the narrow end being slightly larger than typical pipette tips. The wide bore opening at the delivery end is generally between 2 and 5 mm in internal diameter. Further, the wide bore pipette tip can have a hub capable of fitting a standard pipettor or robotic liquid handler.

As used herein, the term “sample buffer” or “sample solution” refers to a solution that contains the analyte of interest, typically with other contaminants. For example, a sample solution may have cells that contain the DNA of interest along with other contaminating cellular material, or a sample solution may have a PCR amplicon of interest along with contaminating primers.

As used herein, the term “wash buffer” or “wash solution” refers to a solution that is used to wash unbound analyte or cell debris from the solid phase substrate—in this case silica.

As used herein, the term “elution buffer” or “elution solvent” refers to a solution that is used to unbind nucleic acid or other analyte from the solid phase substrate—in this case silica.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention.

Herein, any claim introduced with the word comprising is also included under the closed “consisting of” and the semi-closed “consisting essentially of.” However, each claim is not repeated three times in the interest of brevity.

The following abbreviations are used herein:

| ABBREVIATION | TERM |
|---|---|
| DNA | Deoxyribonucleic acid |
| ID | Inner diameter |
| OD | Outer diameter |
| PCR | Polymerase chain reaction |
| RLH | Robotic liquid handler |
| RNA | Ribonucleic acid |
| SPE | Solid phase extraction |
| SPRI | Solid phase reversible immobilization |
| ToT | Tip-on-tip |
| NGS | Next generation sequencing |
| PEG | Polyethylene glycol |
| GMF | Glass microfibers |

### BRIEF DESCRIPTION OF THE DRAWINGS

FIG. 1A-B display a cross-section of a SPRI tip with sintered glass beads (A) and glass microfibers (B).

FIG. 2 is a table providing recoveries of DNA extracted with different diameters of SPRI tips with glass microfibers having a range of 5-20 μm (average 12 μm) in porosity.

FIG. 3A-B shows a cross-section of a SPRI tip using cartridges with sintered glass beads (A) and glass microfibers (B).

FIG. 3C shows a tip with a support disc having a pair of cross hatched support bars.

FIG. 3D shows a tip with an annular support ledge on an inner surface.

FIG. 4 displays DNA recoveries of four DNA fragments with increasing numbers of aspiration/dispensation cycles.

FIG. 5 displays the fluorescently stained agarose gel results of PCR reactions without (lane 1) and with (lanes 2-5) SPRI tip cleanup. Notice the removal of primers and primer dimers in the treated reactions.

### DETAILED DESCRIPTION

Provided herein is a more straightforward way to perform nucleic acid purification without using magnetic beads, wherein pipette tips including silica plugs that are calibrated to allow high flowthrough without the need for centrifugation or high pressures. The pipette tip allows for solutions to pass in and out of the tips while providing reversible immobilization of analytes, such as nucleic acid, for further processing and analysis. We refer to these solid-phase reversible immobilization tip products as “SPRI tips” herein.

The pipette tips used for high throughput in automated procedures are often very narrow in order to work with 384 well plates. The narrow ends approach 0.2 mm in diameter for some robotic tips, so having any material inside the tip would greatly restrict flow. Indeed, there already exists pipette tips with sintered glass bead disks therein for analysis. However, these tips have low flow and either high pressure differentials or centrifugation is required for sufficient flow through the glass disks.

A low porosity silica material, such as 1 μm porosity glass microfiber (GMF), provides the highest recovery for DNA extraction. However, such low porosity material is readily clogged by biological or viscous samples. In fact, 3 μm porosity material restricts flow too much, and viscous DNA sample solutions containing PEG require too much pressure to pass through the material.

Instead, a silica material that is about 5-20 μm in porosity works much better, providing a suitable balance between flow and clogging but still providing significant analyte binding. With a dimension of at least 0.5 mm diameter, or preferably about 0.8-2.0 mm or about 0.8-1.2 mm diameter, and a thickness of approximately 0.5-2.5 mm or 0.5-2.0 mm, good recoveries of e.g., DNA can be achieved with negligible back pressures. Using flow rates of the robotic liquid handler of approximately 5 μL/s to 20 μL/s, the sample, wash and elution solvents flow easily through the silica plug with essentially no restriction. Thus, the SPRI tips described herein can be used with robotic liquid handlers, obviating the need for many tip changes, centrifugation, moving of magnets, magnetic bead contamination and the like.

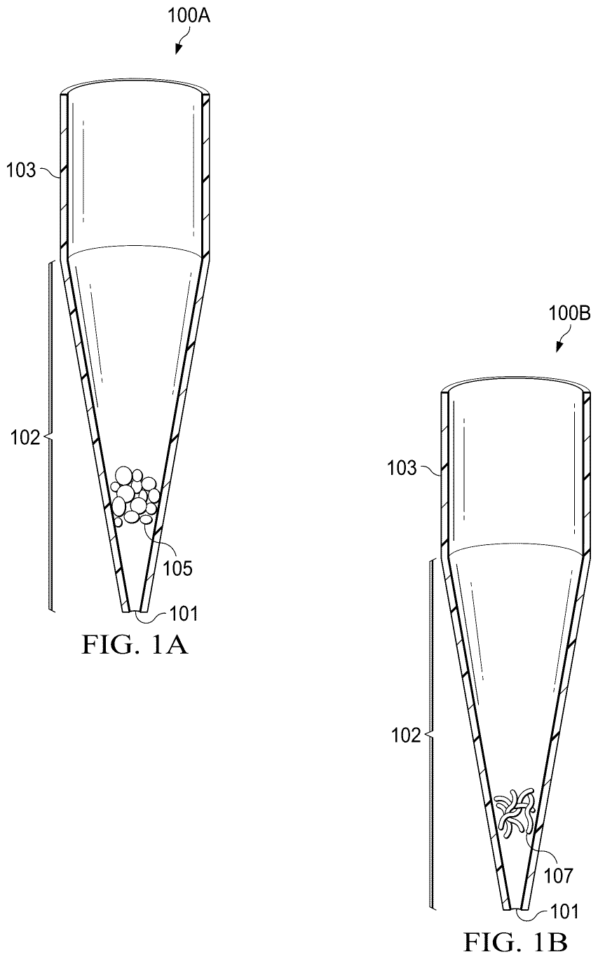

FIG. 1A-B shows a general cross-section of SPRI tips 100 used on a 384 robotic head (not shown) via hub 103. The silica material is positioned in the tapered portion 102 toward the narrow delivery end of the pipette tip 101, where the outer diameter of the silica makes physical contact with the inner wall surface of the pipette tip. FIG. 1A is the SPRI tip with sintered glass beads 105, and FIG. 1B has a glass fiber plug 107.

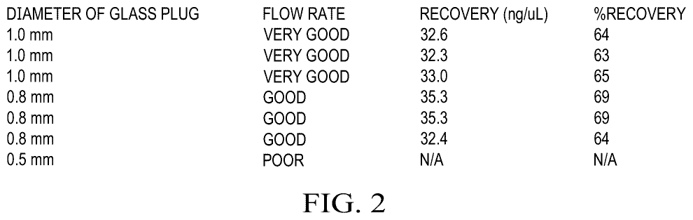

A number of prototype SPRI tips using various diameters of glass plugs were made with GMF of porosities between 5 and 20 μm. These were then tested with a robotic liquid handler set at a flow rate of 10 μL/s using a DNA molecular weight marker VIII test solution. The results are shown in FIG. 2.

If the GMF has a diameter of approximately 0.5 mm, the flows were poor when tested using a robotic liquid handler aspirating at a flow rate of 10 μL/s resulting in an actual flow of about 5 μL/s. Aspirate and dispense cycles had to be greatly increased in time to permit solutions to pass through the GMF at this diameter of glass plug. For the 20 aspirate-and-dispense steps needed for adequate binding, the time was too long, and we thus abandoned this diameter of glass plug.

Using 0.8- and 1.0-mm diameter GMF, however, we found very acceptable flow rates with the flow rate of the robotic liquid handler set at 10 μL/s. The 0.8 mm diameter GMF gave comparable recoveries to the 1.0 mm, and the time was only slightly increased from the 1.0 mm diameter (i.e., a couple of seconds added for a delay). At 1.0 mm, the flow rate at 10 μL/s did not appear to be impeded whatsoever.

One of the issues with these SPRI tips, whether GMF or sintered glass beads, is that it is difficult to have consistent friction fitting of the glass plug in the tips due to the taper of the pipette tips. The sintered glass bead disc is hard to level in the tip, and GMF, which is very random in structure, is difficult to consistently cut and reproducibly fit into each SPRI tip. Even with such challenges, however, we have not noticed an impact with recoveries of DNA in our studies.

Further, there are means of addressing the fit issue, namely sintering beads in a tapered mold to precisely fit the tip, or trimming the disk after manufacture, or lightly pressing the glass fibers into a tapered mold before installation. However, a simpler solution may be to provide a ledge for the sintered beads or GMF or to provide a cartridge for housing same.

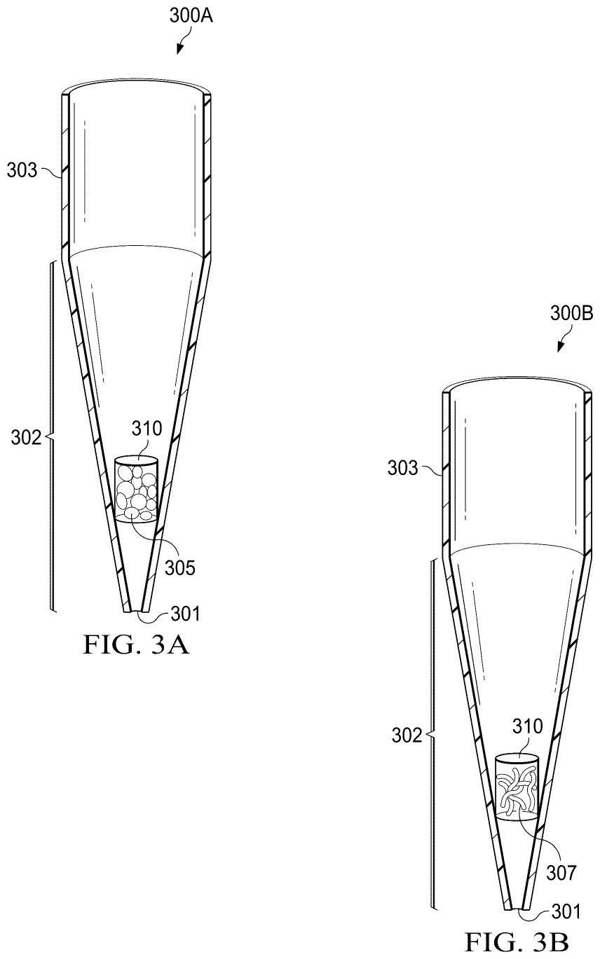

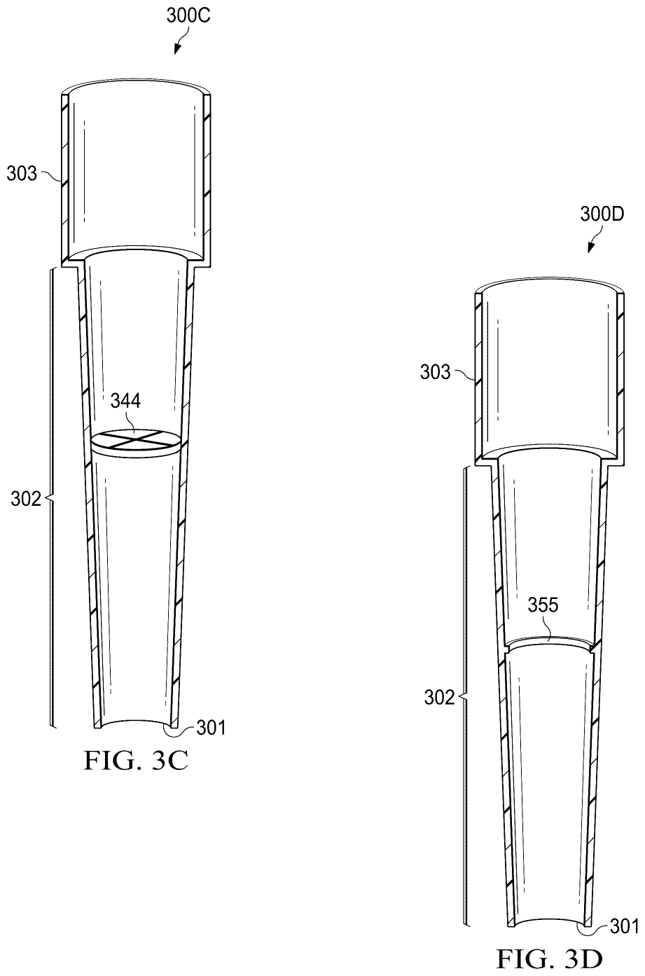

FIG. 3A-B shows a general cross-section of a SPRI tip 300 with a cartridge 310 containing sintered glass beads 305 (A) or GMF 307 (B). The delivery end is 301, and hub end 303, and the silica is present in the tapered portion 302. The cartridge must be made of a strong enough material such that it can be press-fitted to robustly hold in place inside of the pipette tip.

In FIG. 3, the cartridge is shown as cylindrical without a taper, such that the glass beads or GMF can be reproducibly positioned, and friction fitted therein. This would allow for improved robustness. Most importantly, this design allows for a more universal manufacturing capability, such that the cartridge can be placed inside of essentially any pipette tip and the glass plug material modified as needed for the application. Although the pipette tips have a slight taper, with a short length cartridge, there are no losses of liquid due to the inherent dead volumes associated with the top of the cartridge with the pipette tips. However, the cartridge may instead be tapered to fit the tip.

We have made prototype cartridges of GMF and sintered glass beads (app. 100 μm in diameter) using stainless steel tubes with 1.0 mm outer diameter (OD) and 0.85 mm inner diameter (ID) and confirmed their functionality. However, in the future we anticipate evaluating other tubes such as plastic or glass or ceramic. The cartridges must be hard enough for press-fitting with a small OD with the highest ID possible to prevent restricted flow (e.g., >0.5 mm).

The outer diameter of the cartridge needs to be approximately 1.0 mm (app. 0.9 mm to 1.2 mm) at the low end in order to be used with most pipette tips for robotic liquid handlers. The inner diameter needs to be as large as possible but cannot approach OD too closely in order for the cartridge to remain rigid. The cartridge also needs to be short enough to prevent a gap between the top (distant from the narrow end) of the cartridge and pipette tip that may cause losses in liquid from forming, as the bottom end will be the point of contact with the pipette tip. The length must also cover the length of the silica material. and we have found that 2.0 mm works quite well, with a range of 0.2 mm to 2.5 mm being acceptable.

FIG. 3C shows another solution wherein the tip 300C is fitted with a support disk 344 that can be engineered with a taper and thus tightly fit in the tip near the delivery end 301. The support disk 344 is a hollow circle with one or more cross supports, in this case two crossed supports shown. Numerals are otherwise the same as in FIG. 3A-B. FIG. 3D shows a tip with an annular ledge 355 which can be used to support a sintered disc.

It may also be possible to make the SPRI tips with monolithic silica material, or even monolithic material containing carboxylate groups, but these materials have not yet been evaluated herein. It is presumed that these monolithic materials will also have to comply with a range of porosities between 5 μm to 20 μm.

It should also be noted that the silica could be chemically treated to make the silanol groups carboxylate groups. In this case, the porosities and dimensions of the GMF and sintered glass beads would remain the same as previously described. At this time, we have not found any advantage of using carboxylate groups over silanol for these extractions.

### Examples

Prototype glass fiber plug tips (SPRI tips) were made from Hamilton 50 μL pipette tips using 5-20 μm porous GMF having a diameter of 0.8 mm and thickness of about 2 mm. The GMF plug was about 200 μg of fiber and was pushed down into the tip with a blunt end cylinder and friction fitted into place. Large scale production can be done with punch tooling either with or without the use of cartridges. It should be noted that sterilization can be performed using for example an autoclave, but this was not needed for these proof of concept studies.

In this first experiment, we sought to determine herein how many in-and-out aspirating-and-dispensing steps were needed for the most efficient binding of DNA to the glass. An aqueous sample solution containing 1 μg DNA molecular weight marker VIII (25 μL) was diluted with 20 μL binding buffer (30 mM Mg acetate and 20% PEG) and mixed by aspirating-and-dispensing the solution 10-50 times. The solution was subsequently aspirated and dispensed in-and-out of the SPRI tip using a Hamilton Nimbus96 robotic liquid handler. After multiple aspirating and dispensing steps, the SPRI tip aspirated and dispensed a wash solvent (40 μL) once, and then the elution of DNA was performed by aspirating and dispensing the elution solvent (25 μL of 10 mM Tris pH 9 buffer) two times.

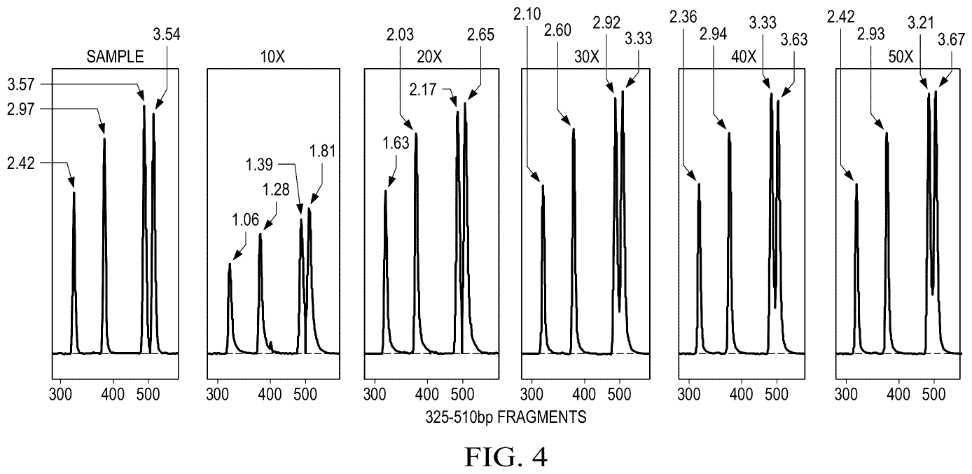

The DNA quantitation results, using a Bioanalyzer (fluorescence detection), are shown in FIG. 4 and demonstrate that increasing the number of aspirating-and-dispensing steps increases the yield, with 50 times providing recoveries of the 4 fragments, left to right, 100%, 99%, 90%, and 103%. However, highly efficient binding was had with fewer in-and-out steps. Thus, efficient DNA preparation can be achieved where the solid-phase reversible binding uses multiple aspirating-and-dispensing steps, preferably at least 20 aspirating-and-dispensing steps, or 30 or 40 or even more.

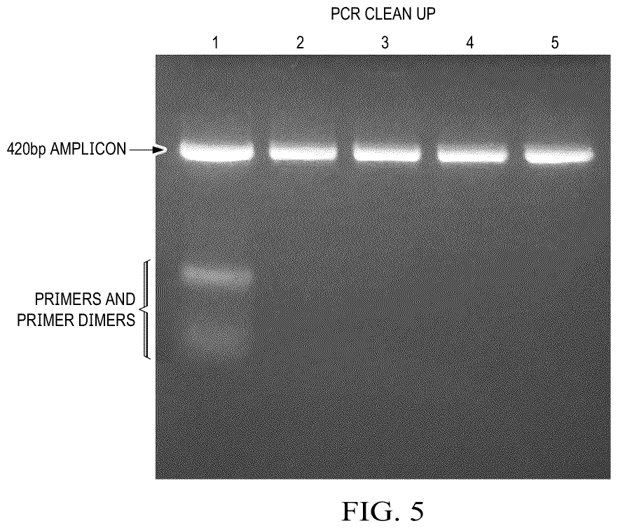

In a second test, we sought to show that very small DNA fragments could be removed from the main amplicon DNA of interest. The SPRI tips were used to cleanup a PCR reaction in order to remove primers and primer dimers from the reaction mixture. The results of the study (N=4) are shown in a gel electropherogram in FIG. 5. Lane 1 is the PCR reaction with amplicon and primers/primer dimers. Lanes 2-5 are the elution after SPRI tip clean up with the removal of the primer/primer dimers and capture of the amplicon.

The following references are incorporated by reference in their entirety.
U.S. Pat. No. 11,193,930 Automated protein precipitation and/or dispersive solid phase extraction using filter tips.
US20090223893 Method and device for desalting an analyte.
US20200025755 Automated protein precipitation and/or dispersive solid phase extraction using filter tips.
US20220184525 Automated group separations of complex mixtures with gel filtration pipette tips.
US2022088537 Membrane devices for filtration and extraction.
US20230075131 US2021373006 Automated solid phase extraction using filter tips.
USD782693 Dispersive insert for pipette tips.
Boom, R. et al., Rapid and simple method for purification of nucleic acids, J., Clin. Microbiol. 28 (3): 495-503 (1990). doi: 10.1128/jcm.28.3.495-503.1990.
Vogelstein, B, et al., Preparative and analytical purification of DNA from agarose, P.N.A.S. 76 (2): 615-619 (1979), https://doi.org/10.1073/pnas.76.2.615.

1. A pipette tip for extracting nucleic acid, said pipette tip comprising:
a) a hub that fits over a pipette head of a separate robotic liquid handler;
b) said hub fluidly connected to a tapered portion fluidly connected to a delivery end;
c) said tapered portion having friction fitted therein a glass plug having a diameter greater than 0.5 mm, and a thickness of between 0.2 to 2.5 mm; and
d) said glass plug comprising a silica material having porosities of 5 to 20 μm.

2. The pipette tip of claim 1, wherein said glass plug has a diameter of 0.8-1.2 mm.

3. The pipette tip of claim 1, wherein said glass plug has a thickness of 0.2-2.0 mm.

4. The pipette tip of claim 1, where said glass plug material is selected from common silicate glass, flint glass, soda lime glass, borosilicate glass, quartz glass, binder-free borosilicate glass, binder containing borosilicate glass, or sintered glass beads or microfiber forms thereof.

5. The pipette tip of claim 1, where silanol groups of said silica material are chemically treated to make active carboxylate groups.

6. The pipette tip of claim 1, where said silica material comprises sintered glass beads.

7. The pipette tip of claim 6, where said glass beads have diameters in the range of 20 to 500 μm.

8. The pipette tip of claim 1, where said silica material comprises glass microfibers.

9. The pipette tip of claim 1, wherein said silica material is contained inside a cylindrical cartridge housing said silica material.

10. The pipette tip of claim 1, wherein said silica material is contained inside a tapered cylindrical cartridge housing said silica material.

11. The pipette tip of claim 1, wherein said silica material is supported on either an annular ledge or a crossed beam support structure.

12. A pipette tip for extracting nucleic acid, said pipette tip comprising:
a) a hub that fits over a pipette head of a separate robotic liquid handler;
b) said hub fluidly connected to a tapered portion fluidly connected to a delivery end;
c) said tapered portion having friction fitted therein a glass plug having a diameter of about 0.8-1.2 mm, and a thickness of about 0.2-2.5 mm; and
d) said glass plug comprising sintered glass beads or glass microfiber having porosities of 5 to 20 μm.

13. The pipette tip of claim 12, where said sintered glass beads or glass microfiber comprises a glass material selected from common silicate glass, flint glass, soda lime glass, borosilicate glass, quartz glass, or binder-free borosilicate glass.

14. The pipette tip of claim 12, where said sintered glass beads have diameters in the range of 50 to 400 μm.

15. The pipette tip of claim 12, wherein said glass plug is inside a cylindrical cartridge housing said glass plug or a tapered cylindrical cartridge housing said glass plug.

16. A method of extracting nucleic acid from a sample, the method comprising the steps of:
a) obtaining a sample container having therein a sample containing nucleic acid in a silica binding buffer;
b) aspirating said sample into a pipette tip of claim 1 and dispensing, said sample back into said sample container a plurality of times until nucleic acid has bound to said silica material, and finish by dispensing said sample into said sample container;
c) aspirating a wash solvent into said pipette tip and dispensing said wash solvent into a waste container and optionally repeating a number of times; and
d) aspirating an elution solvent into said pipette tip and dispensing said elution solvent into a nucleic acid container and optionally repeating a number of times.

17. A method of extracting nucleic acid, the method comprising the steps of:
a) preparing a sample by mixing salt and PEG with a solution comprising nucleic acid in a sample container and allowing said sample to equilibrate;
b) aspirating said sample into a pipette tip of claim 1 and dispensing said sample back into said sample container a plurality of times until said nucleic acid has bound to said silica material in said pipette tip, and finish by a last sample dispensing into said sample container;
c) aspirating a wash solvent into said pipette tip and dispensing said wash solvent into a waste container;
d) optionally repeating step c) a plurality of times;
e) aspirating an elution solvent into said pipette tip and dispensing said elution solvent into a nucleic acid container; and
f) optionally repeating step e) a plurality of times.

18. The method of claim 16, wherein said nucleic acid is DNA.

19. The method of claim 16, wherein said nucleic acid is a PCR amplicon.
